(19) United States
(12) Patent Application Publication
(10) Pub. No.: US 2024/0167061 A1
(43) Pub. Date: May 23, 2024

(54) NANOPARTICLES-MEDIATED CRISPR-CAS9 FOR GENE THERAPY

(71) Applicant: Taipei Veterans General Hospital, Taipei City (TW)

(72) Inventors: Shih-Hwa Chiou, Taipei City (TW); Shih-Jie Chou, Taipei City (TW); Yueh Chien, Taipei City (TW); Yi-Ping Yang, Taipei City (TW)

(73) Assignee: Taipei Veterans General Hospital, Taipei City (TW)

(21) Appl. No.: 18/515,028

(22) Filed: Nov. 20, 2023

Related U.S. Application Data
(60) Provisional application No. 63/426,618, filed on Nov. 18, 2022.

(51) Int. Cl. C12N 15/90; C12N 9/22; C12N 15/11; C12N 15/88

(52) U.S. Cl. CPC C12N 15/907; C12N 9/22; C12N 15/11; C12N 15/88; A61K 48/00; C12N 2310/20; C12N 2800/80

(57) ABSTRACT

The present invention is directed to an integrated conceptual strategy for a gene delivery system, using the combination of nanoparticles, CRISPR-Cas9, and the HITI strategy to deliver CRISPR-Cas9 and achieve effective genome editing; wherein the advanced nanoparticles to overcome the limited packaging size of AAV-based vehicles. Also provided is a promising therapeutic solution for the treatment of hereditary diseases via gene therapy

### CROSS REFERENCE TO RELATED APPLICATIONS

This non-provisional application claims the benefit under 35 U.S.C. § 119(e) to U.S. Provisional Application No. 63/426,618, filed on Nov. 18, 2022, which is hereby expressly incorporated by reference into the present application.

### FIELD OF THE INVENTION

The present invention provides a new cell therapy using nanoparticles-mediated CRISPR-Cas9, and the nanoparticles-mediated CRISPR-Cas9.

### BACKGROUND OF THE INVENTION

Hereditary diseases are known to be caused by genetic disorders and are usually considered incurable. Affected by the hereditary mutation of unique genes, the structures of the proteins encoded by mutated genes are defective or absent, therefore impairing the functions of respective organs in these hereditary diseases. For example, inherited retinal diseases (IRDs) and Fabry disease are considered in the category of hereditary diseases. X-linked juvenile retinoschisis (XLRS), a common early-onset IRDs caused by the mutation of the retinoshisin gene, is featured as the distinctive retinal splitting phenotype, which contributes to the central vision loss, splitting of inner retinal layers, retinal detachment, and other abnormalities (Molday et al., 2012; Wang et al., 2002). Leber's hereditary optic neuropathy (LHON) is also an IRD caused by the mutation of the mitochondrial ND4 gene, and is characterized by bilateral loss of central vision and the degeneration of retinal ganglion cells (Bianco et al. 2017). Best disease, another IRD characterized as a juvenile-onset retinal macular degeneration and the loss of central visual capability, is caused by the mutation of the human bestrophin-1 gene (Sun et al. 2002). Fabry disease is an inherited lysosomal storage disorder associated with the lack of a-galactosidase A (GLA), an enzyme that cleaves globotriaosylceramide (Gb3). Fabry cardiomyopathy as the cardiac manifestation of Fabry diseases is characterized by ventricular hypertrophy and conduction abnormalities (Eng et al., 1994). Till now, the majority of these hereditary diseases still lack effective and reliable treatment.

The rise of gene therapy, especially the development of the clustered regularly-interspaced short palindromic repeats (CRISPR)-CRISPR-associated protein 9 (Cas9) system, has gradually gained importance and provided opportunities for the treatment of hereditary diseases (Ran et al., 2013; Schwank et al., 2013). The CRISPR-Cas9 system is able to exert its function through either the non-homologous end-joining (NHEJ) or the homology-directed repair (HDR) pathways. Improving the efficiency of CRISPR-Cas9 editing is critical for the efficacy of gene therapy. However, the HDR strategy is not readily accessible to post-mitotic cells, limiting its availability in the in vivo applications. Compared to HDR, the NHEJ strategy is available and active in both dividing and non-dividing cells. In addition to the CRISPR-Cas strategies, the delivery of CRISPR-Cas9 machinery is another issue. Among all delivery vehicles, adeno-associated virus (AAV) has been approved by the Food and Drug Administration (FDA) to achieve gene delivery for the treatment of an IRD (Gupta et al., 2017). Nevertheless, the limited virus packaging size of AAV and the generation of neutralizing antibodies against AAV has become the challenges in gene therapy using AAV-based gene delivery (Flotte et al., 2000; Peng et al., 2016). Although several efforts have been made to split the transgenes and use two or three single AAV vectors to deliver them, the transduction efficiency of transgenes using split AAV vectors remain much lower than the conventional AAV-based delivery (Patel et al., 2019; Duan et al., 2001).

It is still desirable to develop a new approach with high efficacy and efficiency for gene therapy to overcome the limitations of AAV vectors for the delivery of the gene CRISPR-Cas9 machineries into the target cells.

### BRIEF SUMMARY OF THE INVENTION

Accordingly, the present invention provides a new approach for gene therapy using specific nanoparticles to deliver CRISPR-Cas9 machineries into the target cells to overcome the limitations of AAV vectors.

In the invention, an advanced CRISPR-Cas9 method is designed to meet the demand of genome editing in combination of HITI technology.

In one aspect, the present invention provides a gene delivery system, which comprises nanoparticles-mediated CRISPR-Cas9 carrying CRISPR-Cas9 components, which is obtained by a self-assembled synthetic preparation of Cas9/sgRNA using homology-independent targeted integration (HITI) technology.

In some embodiments of the present invention, conventional or newly created nanoparticles can be employed, including nanodiamond (ND), supramolecular nanoparticle (SMNP), gold nanoparticles and other potential nanoparticles.

In one example of the invention, SMNP is used to deliver the CRISPR-Cas9 components to achieve effective gene knock-in using the HITI strategy.

In one particular example of the present invention, the gene delivery system is SMNP formulation encapsulating 2-cut dDNA pUC57.RS1.
1. In another aspect, the invention provides a gene delivery method for a gene therapy, which comprises:
   a) preparing a Case9/RNA plasmid incorporated into a nanoparticle to obtain a Cas9/sgRNA plasmid⊂SMNPs;
   b) preparing 2-cut dDNA or MC dDNA⊂SMNPs through stoichiometric mixing of DNA and three SMNP molecular building blocks, including CD-PEI, Ad-PAMAM, Ad-PEG;
   c) using HITI-based knock-in of RS1 gene in Rosa26 locus of mouse genome internalized into the cells, wherein the Cas9/gRNA plasmid is transcribed, translated and assembled to form a Cas9/gRNA RNP complex, and navigated by gRNA the complex excises Rosa26 locus target to induce DSB and dDNA to generate a donor template; and
   d) integrating the donor template between DSB through NHEJ repair pathway.

In a further aspect, the present invention provides a method for gene therapy of a hereditary disease in a patient, which comprises delivering to the target cells in the patient a target gene specific to the hereditary via the gene delivery system or the gene delivery method according to the invention.

In some examples of the present invention, the hereditary diseases are X-linked retinoschisis (XLRS), Leber's Hereditary Optic Neuropathy (LHON), Best disease (BD) and Fabry disease.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The drawings presenting the preferred embodiments of the present invention are aimed at explaining the present invention. It should be understood that the present invention is not limited to the preferred embodiments shown. The patent or application file contains at least one drawing executed in color. Copies of this patent or patent application publication with color drawing(s) will be provided by the Office upon request and payment of the necessary fee.

FIG. 1 shows the scheme to deliver CRISPR-Cas9 and to achieve an effective genome editing according to the present invention, including three key technologies involved in the invention, i.e. nanoparticles, CRISPR-Cas9, and the HITI strategy.

FIG. 2 shows the two strategies of the invention. In this example, a self-assembled synthetic supramolecular nanoparticle (SMNP) was used as the nanoparticles. In addition to the nanoparticles, the overall strategy of HITI-based knock-in of gene was also presented.

FIG. 3 shows the validation of HITI-based RS1 knock-in strategy, showing (a) detailed map of 2-cut Donor DNA pUC57.MC.RS1.EF1.SV40 (pUC57.RS1, 4.9 kb); (b) detailed map of Minicircle Donor DNA MC.RS1.EF1.SV40 (MC.RS1, 3.0 kb); and (c) detection of R-Arm & L-Arm Junction by genomic PCR 72 hours after co-transfection of HEK 293T with Cas9/gRNA plasmid & 2-cut or MC dDNA using Lp3k.

FIG. 4 shows the optimization of SMNP formulation encapsulating Cas9/gRNA plasmid, providing (a) the schematic of SMNP-mediated transfection of Cas9/gRNA plasmid into 293T cell line; (b) the gene disruption efficiencies measured by T7E1 assay of 5 different SMNP formulations designed by varying the ratio (w/w) of Ad-PAMAM and CD-PEIl and gDNA which were extracted 48 hours after transfection; and (c) the bar graph summarizing gene disruption efficiencies (%) of 5 formulations analyzed by T7E1 assays. (Data represent the mean±SEM and were plotted from 2 technical replicates. NP4 with highest indel event frequency (red asterisk) was selected for downstream experiments.)

FIG. 5 shows the TIDE analysis of Cas9/gRNA plasmid-induced gene disruption using three different delivery vehicles, i.e. SMNP, Lipofectamine3000 (LP3k) and TransIT-LT1 (TrLT), including (a) representative graphs of TIDE analysis of gene editing performance induced by Cas9/gRNA plasmid using SMNP, TrLT, LP3k as carriers; and (b) bar graph shows indel frequency induced by Cas9/gRNA plasmid delivered by SMNP, LP3k or TrLT, respectively. (Data were collected from three independent experiments and presented as Mean±S.D. *** p<0.001 between different groups determined by one-way ANOVA with Tukey's multiple comparisons test.)

FIG. 6 shows the optimization of SMNP formulation encapsulating 2-cut dDNA pUC57.RS1: including (a) schematic of SMNP-mediated transfection of 2-cut dDNA pUC57.RS1 into 293T cells; (b) GFP signals under fluorescence microscope of 05 different SMNP formulations designed by varying the ratio (w/w) of Ad-PAMAM and CD-PEI. Pictures were taken 48 hours after the transfection. Scale bar=100 μm; and (c) quantitative graph of transfection performances of 05 formulations determined using ImageJ. For each individual transfected well, GFP micrographs were taken from three different non-overlapped fields. Formulation NP3 (red asterisk) which gave rise to ˜50% GFP+ cells was selected for subsequent experiments. Data is represented as mean±SEM.

FIG. 7 shows the characterization of Cas9/gRNA plasmid⊂SMNPs, including a) hydronamic size of Cas9/gRNA plasmid⊂SMNPs in aqueous media using dynamic light scattering: and b) Transmission electron microscopy (TEM) image of Cas9/gRNA plasmid⊂SMNPs. (Scale bar=200 μm.)

FIG. 8 shows the characterization of MC.RS1⊂SMNPs and pUC57.MC.RS1⊂SMNPs, including (a) Hydronamic size of pUC57.RS1⊂SMNPs in aqueous media using dynamic light scattering: (b) transmission electron microscopy (TEM) image of pUC57.RS1⊂SMNPs; (c) hydronamic size of MC.RS1⊂SMNPs in aqueous media using dynamic light scattering; and (d) transmission electron microscopy (TEM) image of MC.RS1⊂SMNPs. (Scale bar=200 μm.)

FIG. 9 shows the drug loading efficiency evaluated by gel retardation assay, including (a) Cas9/gRNA plasmid⊂SMNPs, and (b) 2-cut dDNA pUC57.RS1⊂SMNPs and MC dDNA MC.RS1⊂SMNPs. 2000 ng of each plasmid was enclosed into SMNPs and subjected to high-speed centrifugation to sediment all SMNPs. 25 ul of upper supernatant was visualized on 1% agarose gel and compared to 400 ng of naked DNA.

FIG. 10 shows the cell-viability assay on LP3K and SMNPs transfection reagents, wherein the effects of LP3K and SMNPs on cell viability of HEK 293T cells after treatment was assessed using the Cell Counting Kit-8 (CCK-8) assay for 48 hours (***, P≤0.001).

FIG. 11 shows the detection of the integration of bacterial backbone template in AAVS2 locus.

FIG. 12 shows the Characterization of RS1/GFP knock-in 293 cells sorted at day 21 post transfection; including (a) bright field and fluorescence images of sorted RS1-knock-in HEK 293T cells after 10 rounds or expansion; (b) R-arm junction and L-arm junction which were successfully amplified from gDNA of sorted 293T cells, signifying the correct integration of gene of interest; (c) sanger sequencing data of these PCR products which were further verified successful RS1 knock-in strategy; and (d) RS1 mRNA expression levels of sorted 293T cells; and (e) representative immunofluorescence images of RS1/GFP-knock-in 293T cells. (Scale bar=50 um.)

FIG. 13 shows the PCR Junction Assay of RS1 integration in AAVS1 locus in transfected HEK 293T cells; wherein (A) a schematic diagram of how PCR primers are designing to detect the exact integration site of RS1/GFP in AAVS1 sequence; (B) two characteristics DNA fragments, i.e., the L-arm and R-arm junctions (677 bp and 721 bp respectively) are detected on the electrophoresis gel, signifying the correct integration of RS1 gene into AAVS1 locus of HEK 293T cells employing CRISPR/Cas9 with donor mc-RS1/GFP plasmids delivered by SMNPs.

FIG. 14 shows the sanger sequencing of PCR products for L-arm and R-arm junctions; wherein sanger sequencing was carried to confirm the corrected DNA sequences of the genome-donor boundaries in the L-arm and R-arm junctions for further validation; RS1 sequence was found integrated in the AAVS1 sequence with minor deletions and insertions at the integration sites.

FIG. 15 provides the schematic illustration of Cas9/sgRNA plasmid and RS1/GFP plasmid transfection in patient retinal organoids via SMNPs delivery platform; depicting that two SMNPs vectors were developed for co-delivery of Cas9/sgRNA plasmid (pXAT2) and mc-RS1/GFP plasmid, enabling CRISPR/Cas9-mediated knock-in of RS1 gene in hiPSCs-derived retinal organoids. After transfecting, the SMNPs vector diffused and resulted in dynamic release of the plasmids into the cells.

FIG. 16 provides the bright-field and fluorescence microscopic images of the Cas9/sgRNA plasmid and mc-RS1/GFP plasmid transfected patient Ros, showing the bright-field and fluorescence images of patient retinal organoids after co-delivery of Cas9/sgRNA plasmid and mc-RS1/GFP plasmid from day 3 to day 40 post transfection; and the GFP expression maintained and gradually diminished after 40 days post transfection.

### DETAILED DESCRIPTION OF THE INVENTION

Unless otherwise defined herein, scientific and technical terms used herein have the meanings that are commonly understood by those of ordinary skill in the art.

The invention provides an integrated conceptual strategy using the combination of nanoparticles, CRISPR-Cas9, and the HITI strategy to deliver CRISPR-Cas9 and to achieve an effective genome editing. This strategy has several advantages, including the higher packing capacity than AAV, precise gene knock-in to replace the defective gene, and the applicability in both dividing and non-dividing cells. Accordingly, the method according to the invention provides a promising therapeutic solution for the treatment of hereditary diseases, such as XLRS, LHON, BD, and Fabry disease.

In the invention, the following three key technologies are used:
(1) CRISPR-Cas9;
(2) Nanoparticles; which are used to deliver the CRISPR-Cas9 components, wherein SMNP us one example of nanoparticles particularly to deliver the CRISPR-Cas9 machineries; and
(3) HITI strategy to achieve effective gene knock-in.

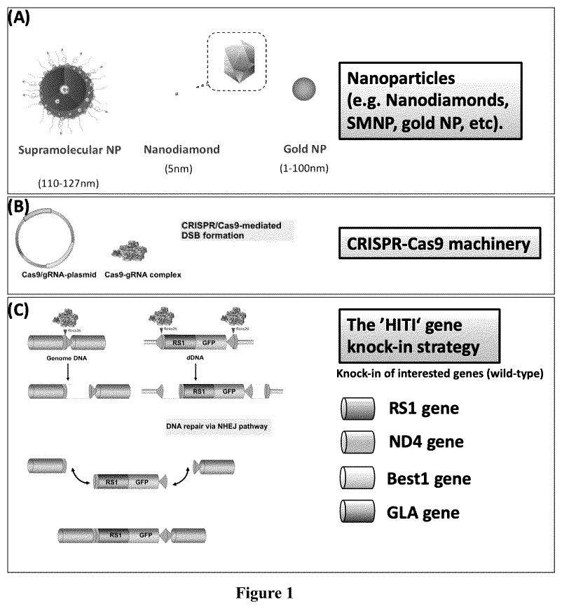

The scheme of the platform according to the present invention is shown in FIG. 1(a), providing the design of SMNP-mediated RS1 targeted integration via HITI.

As shown in FIG. 1(b), the platform comprises two strategies below:
a) A self-assembled synthetic strategy for preparation of Cas9/sgRNA plasmid⊂SMNPs; 2-cut dDNA or MC dDNA⊂SMNPs through stoichiometric mixing of DNA and three SMNP molecular building blocks, i.e., CD-PEI, Ad-PAMAM, Ad-PEG;
b) Overall strategy of HITI-based knock-in of RS1 gene in Rosa26 locus of mouse genome. Upon internalized into the cells, Cas9/gRNA plasmid is transcribed, translated and assembled to form Cas9/gRNA RNP complex. Navigated by gRNA, this complex excises Rosa26 locus target to induce DSB and dDNA to generate donor template. Donor template is then integrated between DSB through NHEJ repair pathway.

HITI Strategy

Homology-independent targeted integration (HITI) is a gene knock-in strategy that can directly ligate foreign DNA to the double-strand breaks via the NHEJ pathway (Auer et al., 2014; Suzuki et al., 2018; He et al., 2016). The HITI strategy is a non-homologous end-joining strategy that can directly insert the foreign DNA into the double-strand breaks (DSBs) in both dividing and non-dividing cells. The steps of the HITI strategy are summarized below:
a) In order to produce minicircle donor DNA (MC dDNA) in the invention, the first step is to construct a parental plasmid that carries relevant sequences required for recombination and minicircle cassette production. The construction of parental plasmid involves of sub-cloning to introduce the cassette, including GFP and human RS1 (hRS1) coding sequence. The parental plasmid was successfully generated and validated by restriction enzyme digestion and Sanger sequencing.
b) Upon arabinose induction, the expression of recombinase is activated, which initiates the recombination of attP and attB sequences to excise the parental plasmid into two smaller circular DNAs. Since parental plasmid and one of these two circular DNAs carry multiple I-SceI sites, they would be subjected to degradation in the presence of endonuclease, which expression is also inducible by arabinose. Meanwhile, only minicircle of interest would remain intact in bacteria and can be extracted and purified following the manufacturer's instructions. The quality and purity of Minicircle DNA MC.RS1 were verified by restriction enzyme digestion, which confirmed a smaller size compared to 7.1 kb-parental plasmid. Also, Sanger sequencing showed attR sequence, which is formed by the joint of attB and attP of the parental plasmid. Altogether, MC.RS1 was produced with satisfactory quality and a sufficient amount for our subsequent experiments.
c) In one example of the present invention, two donor DNAs were used in our HITI-based RS1 knock-in strategy. The first dDNA, in which the insert template is flanked by two cutting sites for targeting gRNA. This plasmid pUC57.RS1 was originally designed and amplified to provide the insert template for the construction of our parental minicircle producer. The second dDNA is newly amplified minicircle dDNA MC.RS1 which harbors only one cutting site.

Nanoparticles

Various nanoparticles with distinct properties have intensively been developed to overcome the limitations of AAV vectors and deliver CRISPR-Cas9 machineries into the target cells. Any conventional or newly created nanoparticles can be used in the invention, including nanodiamond (ND), supramolecular nanoparticle (SMNP), and other potential nanoparticles.

Induced Pluripotent Stem Cell Technologies (IPSC)

In the invention, an advanced CRISPR-Cas9 method was also designed to meet the demand of genome editing for induced pluripotent stem cells, including the patient-derived induced pluripotent stem cells from X-linked retinoschisis (XLRS) patients (Huang et al., 2019), Leber's Hereditary Optic Neuropathy (LHON) patients (Yang et al., 2022), Best disease (BD) (Hsu et al., 2018), and Fabry disease (Chien 2018), which have been developed and obtained.

The invention is further illustrated by the following example, which should not be construed as further limiting.

### Examples

1. Cell Lines and Cell Culture

293T cell line (from ATCC) was maintained in Dulbecco's modified eagle medium (high glucose) supplemented with 10% (vol/vol) heat-inactivated fetal bovine serum (FBS) 1×, 1% (vol/vol) Pen/Strep, 1× GlutaMAX (Gibco), 1×MEM NEAA (Gibco), and incubated at 37° ° C. in a 5% CO2 atmosphere. The cells were regularly tested for Mycoplasma contamination using RT-PCR.

2. Plasmid Construct & Minicircle Production

For plasmid-based CRISPR/Cas9 delivery, plasmid pU6-sgAAVS1-Cas9 carries human codon-optimized SpCas9 sequence driven by CAG promoter and a guide sequence targeting AAVS1 locus driven by U6 promoter. The donor plasmid pUC57.RS1/ND4/BEST1/GLA was designed with EGFP as the reporter gene followed by human RS1/ND4/BEST1/GLA coding sequence, driven by EF1α or CMV promoter. Two cutting sites were introduced to allow for Cas9/AAVS1-targeting gRNA cleavage.

3. Nanoparticle Preparation and Characterization

In the present invention. SMNP was used to deliver the CRISPR-Cas9 machineries. Nanoparticles were prepared by mixing three components Ad-PAMAM, Ad-PEG, CD-PEI in distilled water with varying ratios (w/w), and allowing for nanoparticle self-assembly on ice for at least one hour. Hydrodynamic diameter was measured via dynamic light scattering on a Brookhaven Particle Size Analyzer (Brookhaven) using 150 mM phosphate-buffered saline (PBS) as dilution buffer. Transmission electron microscopy (TEM) images were acquired with a Philips CM120 (Philips Research). The TEM samples were prepared by drop-coating 5 μL of sample suspension solutions onto carbon-coated copper grids and let stand for 5 minutes before excess amounts of solution were removed by filter papers. Subsequently, the samples were negatively stained with 1% phosphotungstic acid (pH 7.2) for 5 minutes before TEM studies.

4. In Vitro Transfection

Lipofectamine 3000 (Life Technologies) or TransIT®-LT1 (Mirus Bio) were used according to manufacturer's instructions. For transfection using nanoparticles, each formulation of Cas9/sgRNA-plasmid⊂SMNPs; 2-cut plasmid dDNA⊂SMNP or Minicircle dDNA⊂SMNPs was added to a well (in a 12-well plate), where 1×10⁵ 293T cells were starved in serum-free DMEM overnight to synchronize the cell cycles to G0/G1 phases. If not specified. 48 hours after transfection, the 293T cells were collected and subjected to genomic DNA extraction or any relevant assay.

5. CRISPR/Cas9 Off-Target Analysis.

To evaluate the off-target effects, the top 5 predicted off-target regions for guide RNA targeting Rosa26 locus were predicted by Cas-OFFinder online algorithm by selecting: SpCas9 from Streptococcus pyogenes, mismatch number ≤3, DNA bulge size=0 and as a target genome of human. Primers were designed accordingly and the surrounding regions were PCR-amplified from genomic DNA of HEK 293T cells transfected with Cas9/sgRNA plasmid using Lipofectamine 3000. To determine the frequency of off-target editing, Sanger sequencing chromatograms were compared with those of a non-transfected group using ICE method as per the manufacturer's instructions.

6. RT-qPCR and qPCR for Genomic DNA.

Total RNA was isolated from harvested cells using Trizol Reagent (Thermo Fisher Scientific) and 4000 ng of RNA was reversely transcribed into cDNA using RevertAid Reverse Transcriptase (Thermo Fisher Scientific) according to manufacturer's standard protocol. An amount of generated cDNA equivalent to 50 ng of RNA was used for qPCR analysis. All reactions were done in triplicates using SYBR green dye on the qPCR system (Applied Biosystem, Foster City, USA). The expression levels were calculated using ΔΔCt method using GAPDH as an internal control.

As for qPCR for genomic DNA, 50 ng of gDNA template was used and reactions were performed in triplicates using SYBR green dye on ABI system. The copy number of DNA targets was normalized to either gGAPDH or AAVS1 locus and compared with control groups.

7. Data Analysis and Statistics

If not specified, all bioassays were performed in triplicates and expressed as mean±S.E.M. The average of data and their respective standard deviation were obtained using Microsoft Excel or GraphPad Prism 7 software. For comparison of two groups, t test (paired or unpaired) was performed by GraphPad Prism 7 software. For comparison of three or more groups, one-way analysis of variance with appropriate multiple comparisons test was analyzed by GraphPad Prism 7 software, P<0.05 was considered statistically significant.

Results

As shown in FIG. 1, the scheme according to the invention comprises three key technologies: nanoparticle technology, CRISPR-Cas9 machinery and the “HITI” gene knock-in strategy.

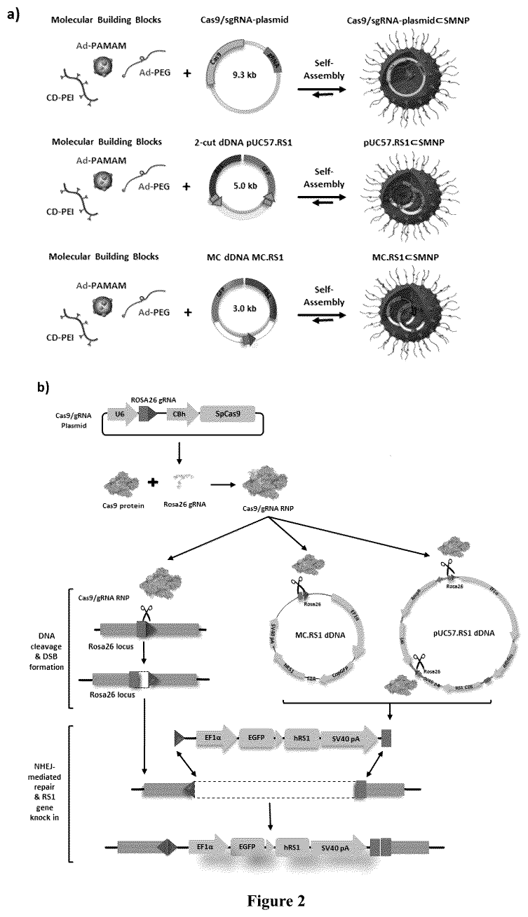

The invention comprises two strategies: As shown in FIG. 2, the two strategies of the invention were presented. In this example, a self-assembled synthetic supramolecular nanoparticle (SMNP) was used as the nanoparticles. In addition to the nanoparticles, the overall strategy of HITI-based knock-in of gene was also presented.

The HITI-based RS1 knock-in strategy was validated. The following constructions were made:
a) 2-cut Donor DNA pUC57.MC.RS1.EF1.SV40 (pUC57.RS1, 4.9 kb);
b) Minicircle Donor DNA MC.RS1.EF1.SV40 (MC.RS1, 3.0 kb).

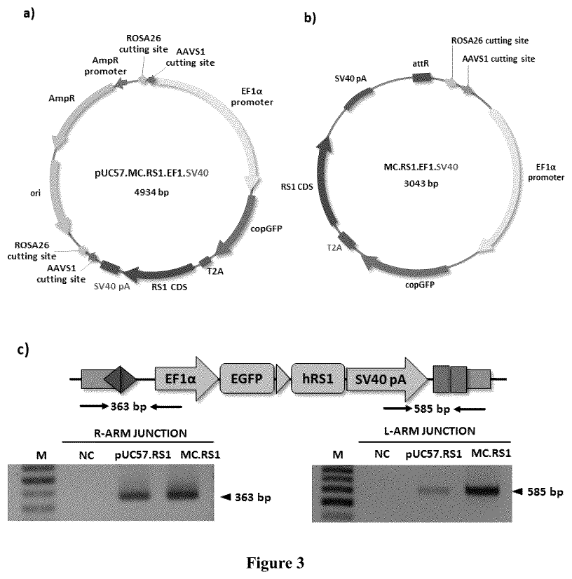

The detailed maps of the constructions are given in FIG. 3. As shown in FIG. 3(c), the detection of R-Arm & L-Arm Junction was resulted by genomic PCR 72 hours after co-transfection of HEK 293T with Cas9/gRNA plasmid & 2-cut or MC dDNA using Lp3k.

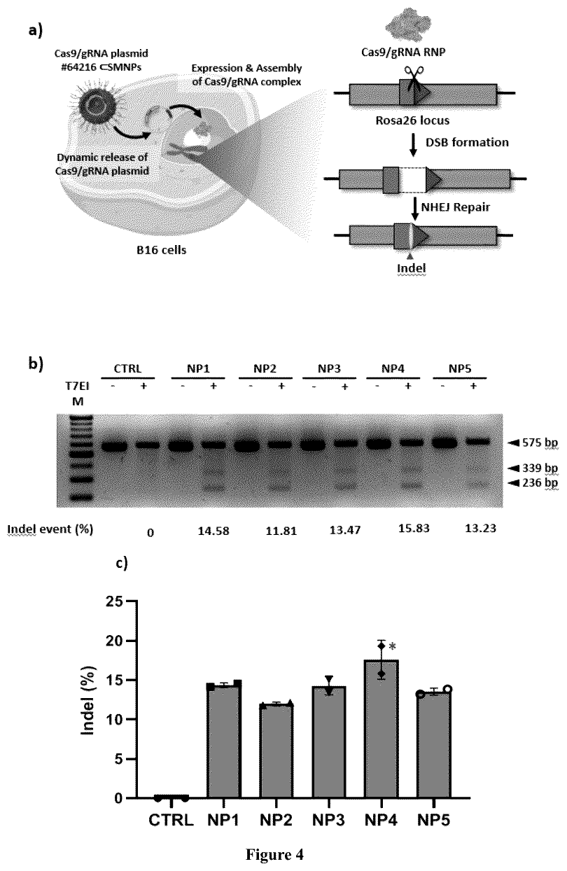

As shown in FIG. 4, the Cas9/gRNA plasmid was encapsulated by SMNP formulation. FIG. 4(a) provides the schematic of SMNP-mediated transfection of Cas9/gRNA plasmid into 293T cell line. FIG. 4(b) shows the efficiencies of the gene disruption as measured by T7E1 assay of 5 different SMNP formulations designed by varying the ratio (w/w) of Ad-PAMAM and CD-PEI. The gDNA was extracted for 48 hours after transfection. The efficiencies (%) of the gene disruption of 5 formulations as analyzed by T7E1 assays were shown in the bar graph in FIG. 4(c). The NP4 with highest indel event frequency (red asterisk) was selected for downstream experiments.

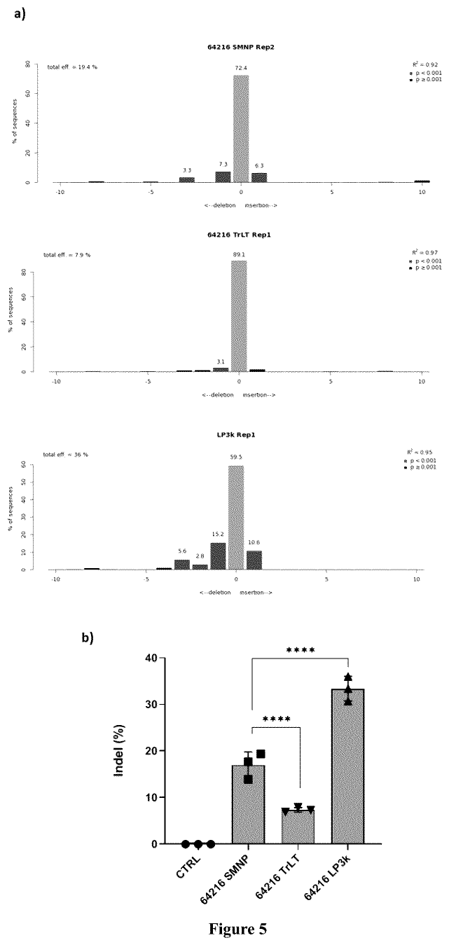

The results of the Cas9/gRNA plasmid-induced gene disruption using three different delivery vehicles. i.e. SMNP, Lipofectamine3000 (LP3k) and TransIT-LT1 (TrLT), were shown in FIG. 5. As shown in FIG. 5(a) showing the results of the TIDE analysis, the gene editing was induced by Cas9/gRNA plasmid using SMNP, TrLT, LP3k as carriers. Indel frequency induced by Cas9/gRNA plasmid delivered by SMNP, LP3k or TrLT, respectively was shown in FIG. 5(b).

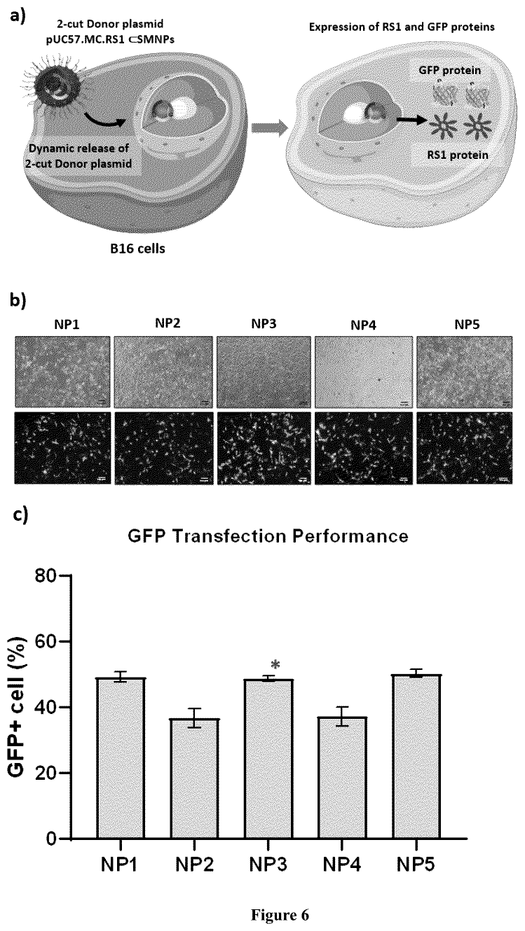

The optimization of 4 was shown in FIG. 6. The SMNP-mediated transfection of 2-cut dDNA pUC57.RS1 into HEK 293T cells was illustrated in FIG. 6(a). GFP signals under fluorescence microscope of 05 different SMNP formulations were designed by varying the ratio (w/w) of Ad-PAMAM and CD-PEI. Pictures were taken 48 hours after transfection, see FIG. 6 b). The quantitative graph of transfection performances of 05 formulations was determined using ImageJ. For each individual transfected well, GFP micrographs were taken from three different non-overlapped fields. Formulation NP3 (red asterisk) which gave rise to ˜50% GFP+ cells was selected for subsequent experiments.

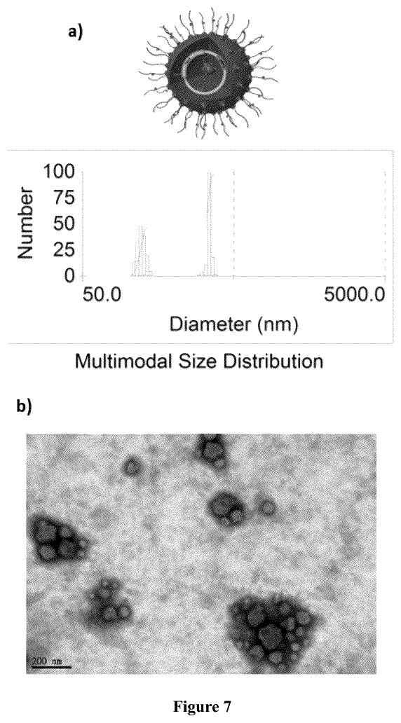

The characterization of Cas9/gRNA plasmid⊂SMNPs was shown in FIG. 7; wherein a) the hydronamic size of Cas9/gRNA plasmid ⊂SMNPs in aqueous media using dynamic light scattering, and b) transmission electron microscopy (TEM) image of Cas9/gRNA plasmid ⊂SMNPs.

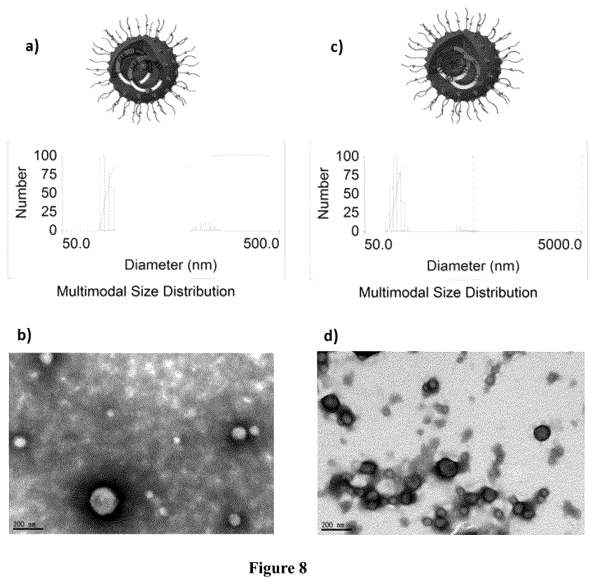

The characterization of MC.RS1⊂SMNPs and pUC57.MC.RS1⊂SMNPs was provided in FIG. 8, showing a) hydronamic size of pUC57.RS1⊂SMNPs in aqueous media using dynamic light scattering: b) transmission electron microscopy (TEM) image of pUC57.RS1⊂SMNPs; c) hydronamic size of MC.RS1⊂SMNPs in aqueous media using dynamic light scattering; and d) transmission electron microscopy (TEM) image of MC.RS1⊂SMNPs.

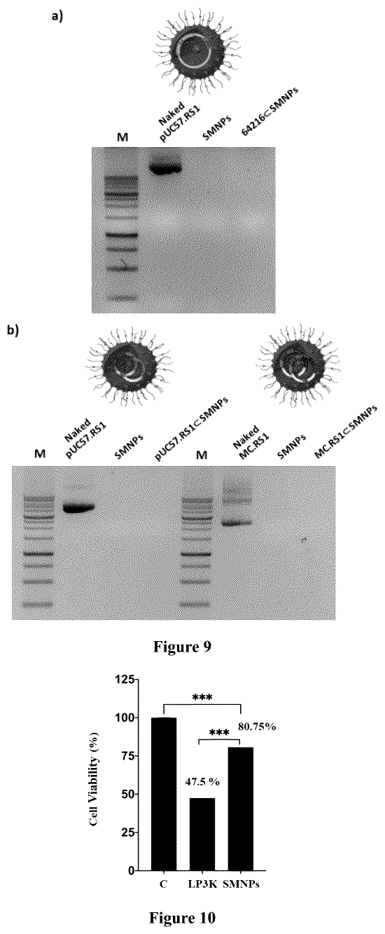

The drug-loading efficiency was evaluated by gel retardation assay for (a) Cas9/gRNA plasmid⊂SMNPs. (b) 2-cut dDNA pUC57.RS1⊂SMNPs and MC dDNA MC.RS1⊂SMNPs, see FIG. 9. 2000 ng of each plasmid was enclosed into SMNPs and subjected to high-speed centrifugation to sediment all SMNPs. 25 ul of upper supernatant was visualized on 1% agarose gel and compared to 400 ng of naked DNA.

The cell viability assay on LP3K and SMNPs transfection reagents was performed, see FIG. 10. The effects of LP3K and SMNPs on cell viability of HEK 293T cells after treatment was assessed using the Cell Counting Kit-8 (CCK-8) assay for 48 hours.

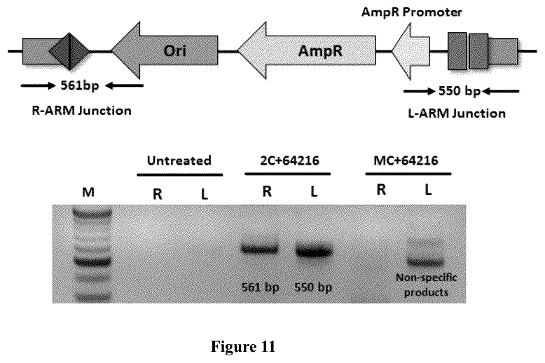

The integration of bacterial backbone template in AAVS1 locus was detected, see FIG. 11. When 2-cut dDNA was used as a repair template, bacterial backbone-associated right and left junction PCR products were successfully amplified from genomic DNA of 293T cells after 3 days of SMNP treatment. On the other hand, no PCR product or only non-specific amplicons are observed in the case of MC.RS1 dDNA.

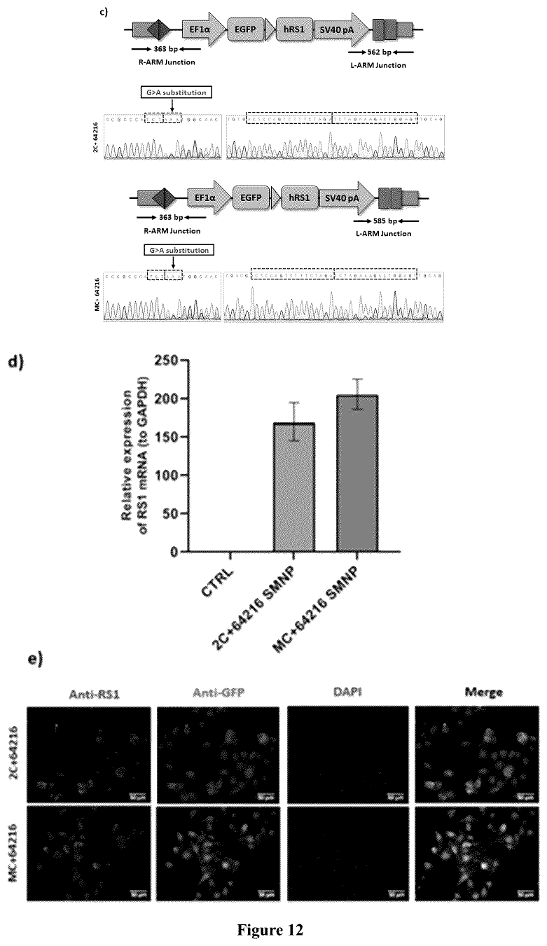

The characterization of RS1/GFP knock-in HEK 293T cells sorted at day 21 post transfection was performed. a) Bright field and fluorescence images of sorted RS1-knock-in 293 cells after 10 rounds or expansion were obtained in FIG. 12 a). R-arm junction and L-arm junction were successfully amplified from gDNA of sorted 293T cells, signifying the correct integration of gene of interest, see FIG. 12 b). FIG. 12 c) provides the Sanger sequencing data of these PCR products further verified successful RS1 knock-in strategy. RS1 mRNA expression levels of sorted 293T cells were shown in FIG. 12 d). FIG. 12 e) provides the representative immunofluorescence images of RS1/GFP-knock-in 293T cells.

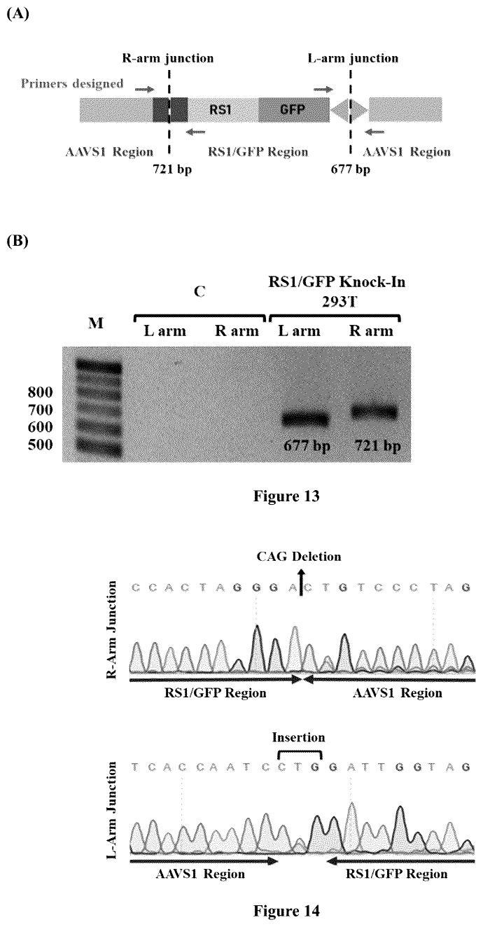

The PCR Junction Assay of RS1 integration in AAVS1 locus in transfected HEK 293T cells was performed. A schematic diagram of how PCR primers are designing to detect the exact integration site of RS1/GFP in AAVS1 sequence, see FIG. 13(A). Two characteristics DNA fragments, i.e., the L-arm and R-arm junctions (677 bp and 721 bp respectively) are detected on the electrophoresis gel, signifying the correct integration of RS1 gene into AAVS1 locus of HEK 293T cells employing CRISPR/Cas9 with donor mc-RS1/GFP plasmids delivered by SMNPs, see FIG. 13(B).

Sanger sequencing of PCR products for L-arm and R-arm junctions was shown in FIG. 14. Sanger sequencing was carried to confirm the corrected DNA sequences of the genome-donor boundaries in the L-arm and R-arm junctions for further validation. RS1 sequence was found integrated in the AAVS1 sequence with minor deletions and insertions at the integration sites.

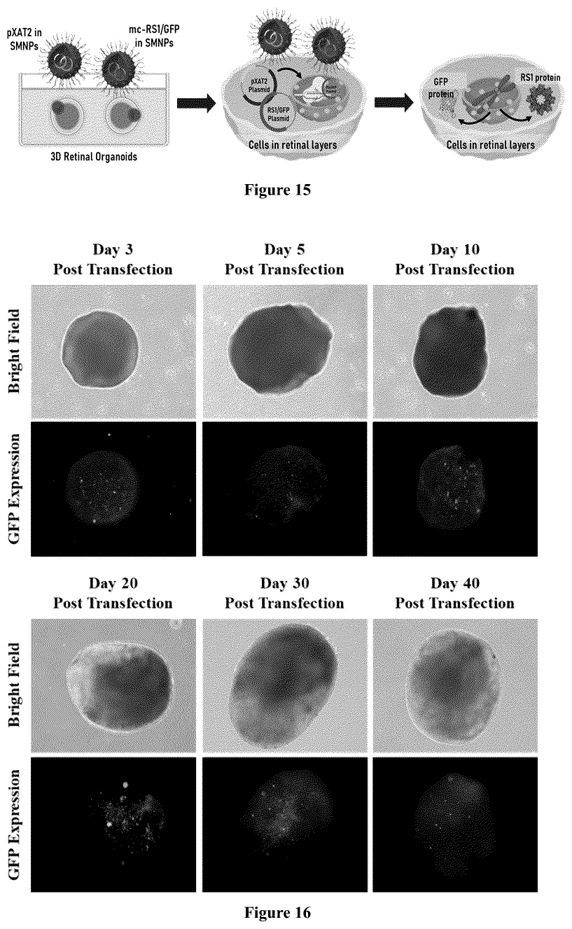

As shown in FIG. 15, the Cas9/sgRNA plasmid and RS1/GFP plasmid transfection could be performed in patient retinal organoids via SMNPs delivery platform. A graphic illustration depicting that two SMNPs vectors were developed for co-delivery of Cas9/sgRNA plasmid (pXAT2) and mc-RS1/GFP plasmid, enabling CRISPR/Cas9-mediated knock-in of RS1 gene in hiPSCs-derived retinal organoids. After transfecting, the SMNPs vector diffused and resulted in dynamic release of the plasmids into the cells.

The bright-field and fluorescence microscopic images of the Cas9/sgRNA plasmid and mc-RS1/GFP plasmid transfected patient ROs were shown in FIG. 16. Bright-field and fluorescence images of patient retinal organoids after co-delivery of Cas9/sgRNA plasmid and mc-RS1/GFP plasmid from day 3 to day 40 post transfection. The GFP expression maintained and gradually diminished after 40 days post transfection.

In summary, our data have provided evidence demonstrating the successful knock-in of the functional genes into precise locus via the combination of nanoparticles, CRISPR-Cas9, and the HITI strategy. Based upon the nature of HITI strategy, this technology is competent to be extended to all in vitro and in vivo conditions. This strategy has several advantages, including the higher packing capacity, precise gene knock-in to replace the defective gene, and the applicability in both dividing and non-dividing cells. Accordingly, the invention provides a promising therapeutic solution for the treatment of hereditary diseases, such as XLRS, LHON. BD, and Fabry disease.

While the present invention has been disclosed by way preferred embodiments, it is not intended to limit the present invention. Any person of ordinary skill in the art may, without departing from the spirit and scope of the present invention, shall be allowed to perform modification and embellishment. Therefore, the scope of protection of the present invention shall be governed by which defined by the claims attached subsequently.

### REFERENCES

Molday R. S., Kellner U., Weber B. H. X-linked juvenile retinoschisis: clinical diagnosis, genetic analysis, and molecular mechanisms. Prog. Retin. Eye Res. 2012; 31:195-212.
Wang T., Waters C. T., Rothman A. M., Jakins T. J., Romisch K., Trump D. Intracellular retention of mutant retinoschisin is the pathological mechanism underlying X-linked retinoschisis. Hum. Mol. Genet. 2002; 11:3097-3105.
Bianco A., Bisceglia L., Trerotoli P., Russo L., D'Agruma L., Guerriero S., Petruzzella V. Leber's hereditary optic neuropathy (LHON) in an Apulian cohort of subjects. Acta Myol. 2017; 36:163-177.
Sun H, Tsunenari T, Yau K W, Nathans J. The vitelliform macular dystrophy protein defines a new family of chloride channels. Proc Natl Acad Sci USA. 2002; 99(6):4008-4013.
Eng C. M., Niehaus D. J., Enriquez A. L., Burgert T. S., Ludman M. D., Desnick R. J. (1994). Fabry disease: twenty-three mutations including sense and antisense CpG alterations and identification of a deletional hot-spot in the alpha-galactosidase A gene. Hum. Mol. Genet. 3 1795-1799.
Ran F A, Hsu P D, Wright J, Agarwala V, Scott D A, Zhang F: Genome engineering using the CRISPR-Cas9 system. Nature protocols 2013, 8:2281-2308.
Schwank G, Koo B-K, Sasselli V, Dekkers J F, Heo I, Demircan T, Sasaki N, Boymans S, Cuppen E, van der Ent C K: Functional repair of CFTR by CRISPR/Cas9 in intestinal stem cell organoids of cystic fibrosis patients. Cell stem cell 2013, 13:653-658.
Gupta P R, Huckfeldt R M: Gene therapy for inherited retinal degenerations: initial successes and future challenges. Journal of neural engineering 2017, 14:051002.
Flotte T R: Size does matter: overcoming the adeno-associated virus packaging limit. Respiratory Research 2000, 1:16-18.
Peng R, Lin G, Li J: Potential pitfalls of CRISPR/Cas9-mediated genome editing. The FEBS journal 2016, 283:1218-1231.
Patel A, Zhao J, Duan D, Lai Y: Design of AAV Vectors for Delivery of Large or Multiple Transgenes. Methods Mol Biol 2019, 1950:19-33.
Duan D, Yue Y, Engelhardt J F: Expanding AAV packaging capacity with trans-splicing or overlapping vectors: a quantitative comparison. Mol Ther 2001, 4:383-391.
Auer T O, Duroure K, De Cian A, Concordet J P, Del Bene F: Highly efficient CRISPR/Cas9-mediated knock-in in zebrafish by homology-independent DNA repair. Genome Res 2014, 24:142-153.
Suzuki K, Izpisua Belmonte J C: In vivo genome editing via the HITI method as a tool for gene therapy. J Hum Genet 2018, 63:157-164.
He X, Tan C, Wang F, Wang Y, Zhou R, Cui D, You W, Zhao H, Ren J, Feng B: Knock-in of large reporter genes in human cells via CRISPR/Cas9-induced homology-dependent and independent DNA repair. Nucleic Acids Res 2016, 44:e85.
Huang K-C, Wang M-L, Chen S-J, Kuo J-C, Wang W-J, Nguyen P N N, Wahlin K J, Lu J-F, Tran A A, Shi M: Morphological and molecular defects in human three-dimensional retinal organoid model of X-linked juvenile retinoschisis. Stem cell reports 2019, 13:906-923.
Yang Y P, Chang Y L, Lai Y H, Tsai P H, Hsiao Y J, Nguyen L H, Lim X Z, Weng C C, Ko Y L, Yang C H, Hwang D K, Chen S J, Chiou S H, Chiou G Y, Wang A G, Chien Y. Retinal Circular RNA hsa_circ_0087207 Expression Promotes Apoptotic Cell Death in Induced Pluripotent Stem Cell-Derived Leber's Hereditary Optic Neuropathy-like Models. Biomedicines. 2022 Mar. 28; 10(4):788.
Hsu C C, Lu H E, Chuang J H, Ko Y L, Tsai Y C, Tai H Y, Yarmishyn A A, Hwang D K, Wang M L, Yang Y P, Chen S J, Peng C H, Chiou S H, Lin T C. Generation of induced pluripotent stem cells from a patient with Best Dystrophy carrying 11q12.3 (BEST1 (VMD2)) mutation. Stem Cell Res. 2018 May; 29:134-138.
Chien Y, Chou S J, Chang Y L, Leu H B, Yang Y P, Tsai P H, Lai Y H, Chen K H, Chang W C, Sung S H, Yu W C. Inhibition of Arachidonate 12/15-Lipoxygenase Improves a-Galactosidase Efficacy in iPSC-Derived Cardiomyocytes from Fabry Patients. Int J Mol Sci. 2018 May 16; 19(5):1480.
Yang T C, Chang C Y, Yarmishyn A A, Mao Y S, Yang Y P, Wang M L, Hsu C C, Yang H Y, Hwang D K, Chen S J, et al: Carboxylated nanodiamond-mediated CRISPR-Cas9 delivery of human retinoschisis mutation into human iPSCs and mouse retina. Acta Biomater 2020, 101:484-494.
Chou S J, Yang P, Ban Q, Yang Y P, Wang M L, Chien C S, Chen S J, Sun N, Zhu Y, Liu H, et al: Dual Supramolecular Nanoparticle Vectors Enable CRISPR/Cas9-Mediated Knockin of Retinoschisin 1 Gene—A Potential Nonviral Therapeutic Solution for X-Linked Juvenile Retinoschisis. Adv Sci (Weinh) 2020, 7:1903432.

1. A gene delivery system for a gene therapy, comprising: nanoparticles-mediated CRISPR-Cas9 carrying CRISPR-Cas9 components, which is obtained by a self-assembled synthetic preparation of Cas9/sgRNA using homology-independent targeted integration (HITI) technology.

2. The gene delivery system of claim 1, wherein the nanoparticle is nanodiamond (ND), supramolecular nanoparticle (SMNP) or gold nanoparticle.

3. The gene delivery system of claim 2, wherein the nanoparticle is SMNP.

4. The gene delivery system of claim 1, which is used to deliver the CRISPR-Cas9 components to achieve effective gene knock-in using the HITI strategy.

5. The gene delivery system of claim 1, which is a SMNP formulation encapsulating 2-cut dDNA pUC57.RS1.

6. A gene delivery method for a gene therapy, which comprises:
e) preparing a Case9/RNA plasmid incorporated into a nanoparticle to obtain a Cas9/sgRNA plasmid⊂SMNPs;
f) preparing 2-cut dDNA or MC dDNA⊂SMNPs through stoichiometric mixing of DNA and three SMNP molecular building blocks, including CD-PEI, Ad-PAMAM, Ad-PEG;
c) using HITI-based knock-in of RS1 gene in Rosa26 locus of mouse genome internalized into the cells, wherein the Cas9/gRNA plasmid is transcribed, translated and assembled to form a Cas9/gRNA RNP complex, and navigated by gRNA the complex excises Rosa26 locus target to induce DSB and dDNA to generate a donor template; and
d) integrating the donor template between DSB through NHEJ repair pathway.

7. A method for gene therapy of a hereditary disease in a patient, which comprises delivering to the target cells in the patient a target gene to the hereditary via the gene delivery system set forth in claim 1.

8. The method of claim 7, wherein the hereditary disease is X-linked retinoschisis (XLRS), Leber's Hereditary Optic Neuropathy (LHON), Best disease (BD) or Fabry disease.
